(19) United States
(12) Patent Application Publication
(10) Pub. No.: US 2025/0041078 A1
(43) Pub. Date: Feb. 6, 2025

(54) LOAD AND GAP BALANCE SYSTEM IN KNEE REPLACEMENT SURGERY

(71) Applicant: Unik Orthopedics, Inc., San Jose, CA (US)

(72) Inventor: Charlie Wen-Ren CHI, Milpitas, CA (US)

(21) Appl. No.: 18/789,010

(22) Filed: Jul. 30, 2024

Related U.S. Application Data
(60) Provisional application No. 63/530,019, filed on Jul. 31, 2023.

(51) Int. Cl. A61F 2/46

(52) U.S. Cl. CPC A61F 2/4657; A61F 2002/4633; A61F 2002/4658; A61F 2002/4666

(57) ABSTRACT

The present invention relates to a system and method for load and gap balancing in knee replacement surgery. The system incorporates sensor devices, such as load cells, pressure, ultrasound or optical, to accurately measure gaps and ligament tensions within the knee joint during surgery. Additionally, a tensioner device, which can be a manual spreader, a spring-loaded mechanism, or a motorized linear actuator, is employed to tension the medial and lateral collateral ligaments, as well as patella tendon, based on the measured data. These real-time measurements are transmitted to a computer or robotic surgery system, which provides real-time visualization of the data and assists the surgeon in determining the appropriate gap and ligament tension for each individual patient. Using soft tissue analysis, the real-time sensor data can be displayed on a computer recommending appropriate gap and ligament tension, implant size and shape, and individualized knee alignment based on optimal outcome.

### CROSS-REFERENCE TO RELATED APPLICATION

This application is related to and claims priority under 35 U.S.C. § 119 (e) from U.S. Patent Application No. 63/530,019 filed Jul. 31, 2023, titled “Load and Gap Balancer System in Knee Replacement Surgery,” the entire contents of which is incorporated herein by reference for all purposes.

### BACKGROUND

Knee replacement surgery is a common procedure performed to alleviate pain and improve mobility in patients with severe knee joint degeneration. However, achieving optimal outcomes can be challenging due to the complexities involved in balancing the extension and flexion gaps and tissue tensions (load) of the medial/lateral collateral, posterior cruciate, and patella ligaments within the knee joint during the procedure. For example, the tension (load) of MCL and LCL for each patient is different. Traditionally, surgeons have relied on manual techniques and subjective assessments to achieve symmetric gap balance and ligament tension. These methods often result in suboptimal outcomes and may lead to complications such as instability, limited range of motion, and premature wear of the artificial knee implants.

### SUMMARY

The system and method for load and gap balancing in knee replacement surgery offer significant advancements in achieving personalized knee alignment and optimal clinical outcomes. By utilizing sensor devices to measure the gaps within the knee joint, surgeons can obtain accurate and objective data regarding the extension and flexion gaps of the femoral-tibial and patella-femoral joints at different degrees of knee motion. The tensioner device allows for precise tensioning of the medial and lateral collateral ligaments based on these measurements.

The system further includes patient-specific knee instruments that determine the initial position and orientation of the knee implants based on preoperative planning. This information serves as a physical reference for the gap balancing device, which calculates the extension and flexion knee gaps accordingly. Real-time data is displayed on a computer monitor, facilitating informed decision-making by the surgeon during the procedure. Subsequently, manual instruments or robotic systems can be used to achieve the desired load and gap for optimal implant positioning.

The system may take the form of a dynamic gap/load balancing device, which measures the three compartments of the knee joint (medial, lateral and patellafemoral) throughout the entire range of motion. Sensor devices embedded in trial implants measure the load and gaps to determine the optimal implant size, thickness and shape of the femoral component, tibial inserts and patella button based on tissue analysis from implant design, bone morphology, and knee alignment. Soft tissue analysis can be used to recommend the optimal combination of implant size and shape to achieve the desired patient outcomes based on real-time data.

The system's communication interface enables seamless data transmission between the sensor device and the computer or robotic surgery system, whether wired or wireless, utilizing technologies such as Bluetooth, Cellular or Wi-Fi. This ensures efficient and reliable data exchange, enhancing the overall surgical experience.

In summary, the load and gap balancing system in knee replacement surgery enable precise measurement of gaps, accurate tensioning of ligaments, personalized alignment, and optimal knee implants. This innovation has the potential to improve surgical outcomes, patient satisfaction, and long-term durability of knee replacements.

One implementation of the present disclosure includes a system for knee replacement surgery. The system may include a sensor measuring a distance of a gap in a knee joint during a knee replacement procedure, a tensioner device providing tension to the medial and lateral collateral ligaments based on the distance measurement obtained from the sensor, and a computing system displaying real-time data from the sensor on a display in communication with the computing system. In addition, a robotic surgical device may be included for shaping a distal end of a femur based on the gap information and on a patient-specific knee instrument mated to the femur to provide an initial position and orientation of a knee implant. The computing system may determine a recommended implant design and an implant size based on dynamic knee measurements received from the sensor and the tensioner device during the knee replacement procedure.

Another implementation of the present disclosure may include a method for knee replacement surgery. The method may include the operations of obtaining a preoperative two-dimensional imaging scan of a patient's knee joint for preoperative planning of the knee replacement surgery, manufacturing a patient-specific instrument for knee replacement surgery based on the preoperative planning, resecting, utilizing the patient-specific instrument, a femur and a tibia of the knee joint, and measuring a gap between the resected femur and the resected tibia of the knee joint at 0 and 90 degrees using a sensor device. The method may also include the operations of determining a position and an initial size of one or more knee implants based on the measured gaps, sending the position and the initial size of the one or more knee implants to a robotic surgical system, shaping, based on the position and the size of the one or more knee implants, the distal end of the femur with the robotic surgical system, and receiving, from a computing device and based on the measured gap from the sensor device, an updated implant size and implant shape based on the measured gap received from the sensor device during the shaping of the distal end of the femur.

### BRIEF DESCRIPTION OF THE DRAWINGS

Example embodiments will become more fully understood from the detailed description given herein below and the accompanying drawings, wherein like elements are represented by like reference characters, which are given by way of illustration only and thus are not limitative of the example embodiments herein.

FIGS. 1A-1B are oblique views of patient-specific instruments pinned to the distal femur with saw guide for making bone cuts in accordance with one embodiment.

FIGS. 2A-2B are oblique views of the patient-specific instruments pinned to the proximal tibia with saw guide for making bone cuts in accordance with one embodiment.

FIGS. 3A-3B show the initial reference of the gap balancing device after primary bone cuts in accordance with one embodiment.

FIG. 4 is a flowchart of a method for a gap and load balancing procedure for knee replacement surgery in accordance with one embodiment.

FIGS. 5A-5B are frontal views of the knee showing extension gap balancing in accordance with one embodiment.

FIGS. 6A-6B are transverse views of the knee showing flexion gap balancing in accordance with one embodiment.

FIGS. 7A-7B are frontal views of the knee showing extension gap balancing using a manual tensioner in accordance with one embodiment.

FIG. 8 is a lateral view of a knee showing flexion gap balancing using a manual tensioner for a single compartment in accordance with one embodiment.

FIG. 9 is a transverse view of a knee showing flexion gap balancing using sensor devices to measure the gap and load for medial compartment in accordance with one embodiment.

FIG. 10 is a transverse view of a knee showing flexion gap balancing using an actuator for the medial compartment and a spring in the lateral compartment for ligament tensioning in accordance with one embodiment.

FIG. 11 is a frontal view of a femur showing a robot mounting assembly with rotational adjustments based on an initial reference from patient-specific instruments in accordance with one embodiment.

FIG. 12 is a transverse view a femur showing a robot mounting assembly with rotational adjustments based on an initial reference from patient-specific instruments in accordance with one embodiment.

FIGS. 13A-13B are transverse views of a robot surgical device mounted on a medial femur with rotational and translational adjustments for making secondary bone cuts of the femur in accordance with one embodiment.

FIG. 14 is a lateral view of a robot surgical device mounted on the medial femur for with flexion and translational adjustments for primary and secondary cuts of the femur in accordance with one embodiment.

FIG. 15 illustrates a knee anatomy with a patella-femoral compartment and patella component including three translation and three rotation motion.

FIG. 16 is a cross-sectional view of a patella component with a six-axis load sensor in accordance with one embodiment.

FIGS. 17A-17B are views of a knee with implant components for dynamic gap and load balancing using sensors for real-time data analysis in accordance with one embodiment.

FIGS. 18A-18B are views of a motorized linear actuator measuring the medial compartment gap and load and transmitting the data wirelessly to a computer displaying real-time data in accordance with one embodiment.

FIG. 19 is a diagram illustrating an example of a computing system which may be used in implementing embodiments of the present disclosure.

### DETAILED DESCRIPTION

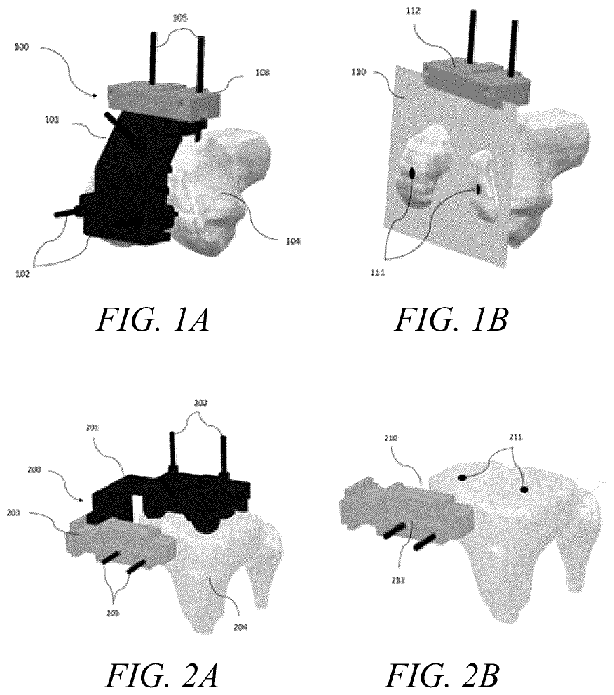

Aspects of present disclosure involves gap and load balancing system for the knee joint including total, uni-condylar, and patella femoral replacement surgeries. FIGS. 1A-1B and FIG. 2A-2B illustrate patient specific femoral and tibial cutting guides, 100 and 200 respectively, to assist the surgeon in resecting the ends of the femur 104 and tibia 204 bones to receive a total knee prothesis. Examples of such patient-specific guides are described in U.S. Patent Application No. US 2016/0038245 entitled “Method for Creating a Customized Arthroplasty Resection Guide Utilizing Two-Dimensional Imaging”, filed on Aug. 6, 2015, the entirety of which is incorporated by reference herein.

In one example of generating a customized arthroplasty resection guide, a patient imaging device may be used to obtain a plurality or series of images of a patient's anatomy, such as a damaged joint. The images may be obtained via an imaging device or system, such as a CT scanner, MRI scanner, X-Ray scanner, or any other type of imaging device. In general, the imaging data or scans may be utilized by a surgeon to generate or approve a surgical plan for the arthroplasty procedure. The pre-operative planning may include a surgeon reviewing the patient scans and selecting one or more steps of the arthroplasty procedure based on the scans, such as cut planes location on the patient's joint to remove the damaged portion of the patient's bone for implanting a joint replacement device, a resurfacing plan of the damaged portion of the patient's bone, an implant location and orientation within the joint, and the like. The pre-operative planning conducted by the surgeon may include, in some instances, transmitting the scanned images or other patient data over a network to a computing device on which the surgeon may view the images. The surgeon may also provide indications or other information of the planned procedure via the computing system on which the images are viewed.

Simultaneously to the surgeon's pre-operative planning, a bone registration device 100 and 200 may be generated based on the patient scans. In some instances, the bone registration device 100 and 200 may be based on a segmentation process performed on the patient scans or may include generating a three-dimensional model of the patient's joint. In other instances, however, the customized bone registration devices 100 and 200 may be generated from a collection of two-dimensional images or scans of the patient. In particular, systems, methods, computer program products, manufacture process and the like, may be provided for as customized arthroplasty registration device 100 and 200 from one or more two-dimensional (2D) images of the patient's joint to undergo the arthroplasty procedure. The method may include receiving the 2D images of the joint from an imaging device, reformatting the images, and creating a customized registration device 1404 template from the images. In general, one or more landmarks may be electronically marked on one or more of the series of 2D images of the patient's joint through a computing device. These electronic markers on the series of 2D images correspond to landmarks of the patient's joint undergoing the arthroplasty procedure. Once the template for the registration device 100 and 200 is created by the computing device utilizing one or more of the electronic markers on the 2D images, a tool path or milling program is generated by the computing device. The tool path or milling program may then be provided to a milling or 3D printing machine to create the registration guide corresponding to the machine-specific program. The registration device is thus customized to the landmarks identified in the series of 2D images of the patient's joint. Further, the procedure does not require the generation of a three-dimensional (3D) model of the patient's anatomy to create the customized nature of the registration device. Rather, by utilizing one or more mating shapes that contact the joint anatomy at particular contact points of the joint anatomy corresponding to the identified landmarks in the 2D images, the customization of the registration device is achieved. Further, because the process does not require the generation of a 3D model, the customized registration guides may be produced more quickly and efficiently than previous customization methods.

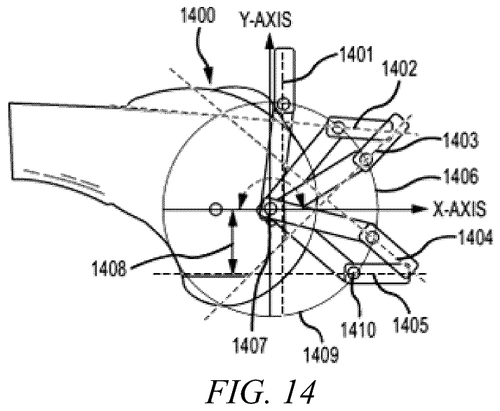

The patient-specific femoral guide 101 and tibial guide 201 illustrated in FIGS. 1A-2B may be generated through the method above and establish an implant position and orientation based on the patient's pre-operative imaging scan. Attached to the patient guides 101 and 201 are saw guides for the femur 103 and tibia 202, which together constitute a bone resection guide for the femur 104 and tibia 204. In particular, FIGS. 1A and 2A illustrate patient guides 101 and 201, respectively, secured to a patient's bone using two or more metal pins or drill bits 102 at the distal femur and at the proximal tibia 202. FIGS. 1B and 2B illustrate the saw guide 103 and 203 secured to the bone using two pins 205. In these examples, an oscillating bone saw may be used to resect the distal femoral condyles 110 and proximal tibial plateau 210 using the saw slots 112 and 212, as illustrated. In some examples, distal pins 102 and proximal pins 202 used to secure the saw guides 103 and 203 may provide visual reference holes approximately 3.2 mm in diameter after the bone resections. The distal plane 110 and two distal holes 111 are sufficient to specify three positions (e.g., Medial Lateral (M/L), Anterior Posterior (A/P) and Proximal Distal (P/D)) and three rotations (e.g., Varus Valgus (V/V), Flexion Extension (F/E) and Internal External Rotation (I/E)) of a femoral prothesis (shown in FIG. 14 and discussed in more detail below) for all three-planes. Similarly, the proximal plane 210 and guide holes 211 are sufficient to specify the three positions and three rotations of the tibial prothesis.

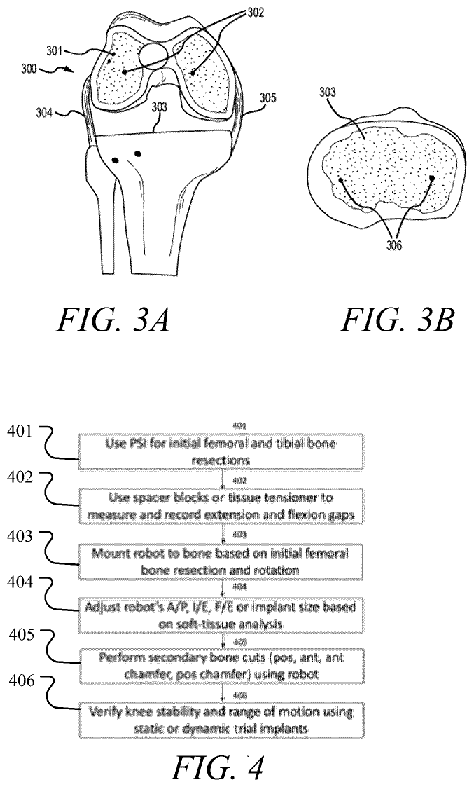

FIGS. 3A-3B illustrate a knee joint 300 after the initial bone resections have been made using patient-specific instruments establishing the initial position and orientation of the femoral and tibial prothesis. As mentioned above, a plane establishes the P/D, F/E and V/V of the femoral 301 and tibial 303 components and the two visual holes 302 and 306 represent the I/E, M/L and AP position of the femoral and tibial components respectively. In general, using the distal femoral holes as reference is more consistent than using other bony landmarks. The medial and lateral posterior condyles are typically arthritic, where the cartilage and subchondral bone have been worn out and using the arthritic bone as reference provides inaccurate measurements of the true gap of the physiologic knee. Thus, by using the bone resection plane and drill holes as line reference, the correlation between the implant position and orientation relative to the physiologic knee can be established.

One of the goals of gap balance is to ensure the medial collateral ligament (305) and lateral collateral ligament (304) are tensioned properly after implantation for optimal outcome. A secondary goal is to ensure enough bone has been removed based on the implant design and sizes. For example, the thickness of the femoral component can range from 8 to 10 mm both distally and posteriorly and 8 to 18 mm in 1- or 2-mm increments for the tibial component. For the femoral component of thickness 8 mm and tibial component of 8 mm to fit within the knee's soft tissue envelope, the extension and flexion gaps should be at least 16 mm. Also, the extension and flexion gaps in the native knee varies by compartment. For example, under 100 Newton force per compartment, the extension gap is ˜6 mm medially and ˜7 mm laterally for knees in physiologic conditions. Additionally, as part of the total knee replacement procedure, the anterior cruciate ligament (ACL) and/or posterior cruciate ligament is sacrificed thus increasing the extension and flexion gaps by 2-5 mm. Typically, the gap in the lateral compartment is larger than the medial compartment due to the size (length, width, and thickness) and bone morphology (epicondyles).

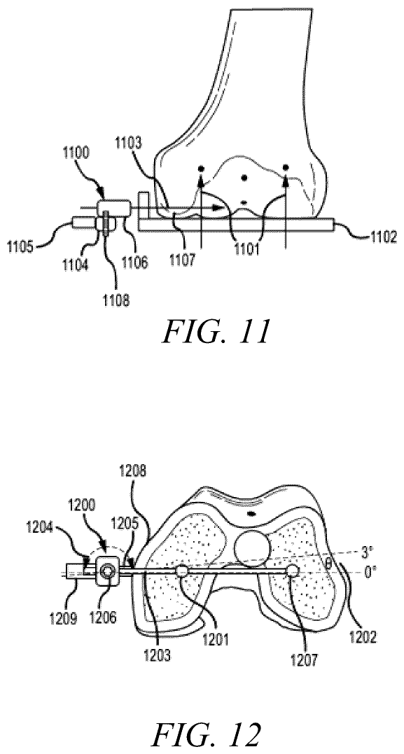

FIG. 4 is a flowchart describing the procedural steps for the gap balancing system with a bone-mounted knee robot. For more details of the bone-mounted robot, reference is made to US Patent Application No. US20210137613A1 “Methods and Systems for Robotic-assisted Surgery Using Customized Bone Registration Guides”, the entirety of which is incorporated by reference herein. Starting with operation 401, the patient-specific femoral and tibial resection guides described in FIGS. 1A-1B and 2A-2B are used to establish the initial reference planes, 301 and 303, and line from two distal holes 302. Next in operation 402, spacer blocks of different thicknesses, like the available implant (femur and tibia) thicknesses or tissue tensioner, is used to measure and record the extension and flexion gaps based on a certain tension using the initial reference established in operation 401. In operation 403, the robot's mounting fixture is attached to the bone using same initial reference as the gap balance system near the anterior medial femur as shown in FIGS. 11-12 below. By using the same bony reference as the gap balance system, the measured data can be used to adjust the robot's cutting position and orientation (A/P, I/E, F/E, or implant size) to achieve the desired extension and flexion gaps in operation 404. In operation 405, the surgeon uses the robotic-assisted system for making the secondary bone cuts of the femur as shown in FIG. 14 below. In operation 406, the surgeon verifies the knee stability and range of motion using static or dynamic trial implants similar to the final from the implant manufacturer. The purpose is to ensure that the extension and flexion gaps are balanced throughout the entire knee range of motion. Different tibial insert thicknesses can be used as a tensioner to balance the MCL and LCL. In addition, a patella load sensor can be used to determine the dynamics of the patella femoral joint as described in more details in FIG. 17.

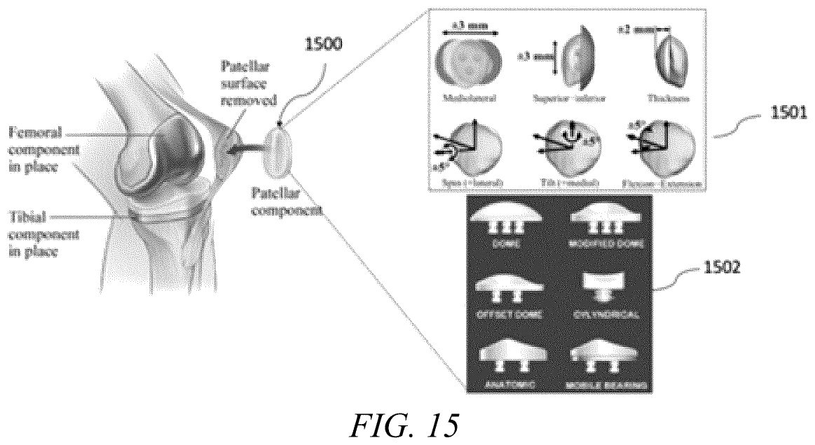

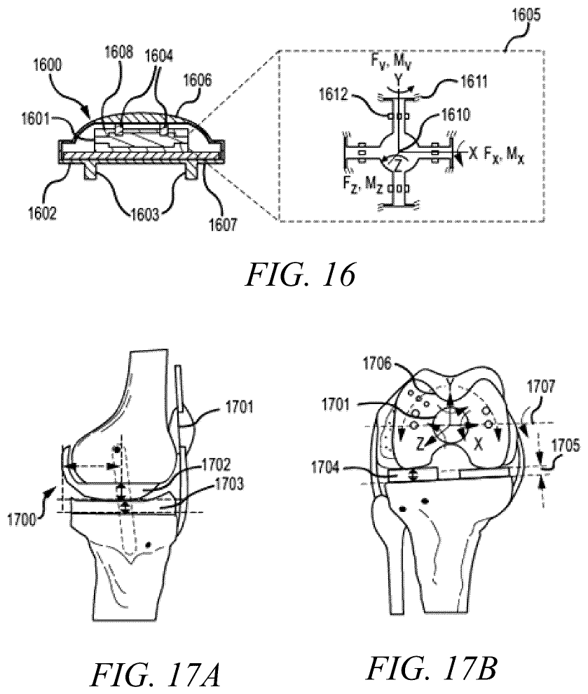

One of the goals of total knee surgery is to achieve an equal and symmetric extension and flexion gaps as shown in FIGS. 15-16. In a standard TKA, the goal is to achieve a neutral mechanical knee alignment (0 Degree) in the frontal plane when the knee 500 is extended. For example, a reference line is established from the center of the hip joint to the center of the knee to the center of the ankle joint using X-ray/MRI/CT. The distal femoral and proximal tibial bone resection are then perpendicular to the neutral mechanical reference line. Taking into consideration the implant thicknesses, the resection plane can be established for the distal femur 501 and proximal tibia 502. The amount of bone removed in the medial 505-506 and lateral 503-504 compartments may be the same or greater than the implant manufacturers available thicknesses to achieve a rectangular extension gap 514. In most implant designs, the combined implant thickness in extension and flexion gaps are the same. Therefore, it is desirable to achieve a symmetric medial 511 and lateral 510 gaps while achieving the desired ligament balance of MCL 513 and LCL 512. A spacer block of the same thickness as the combined implant thickness can be used to assess the extension gaps.

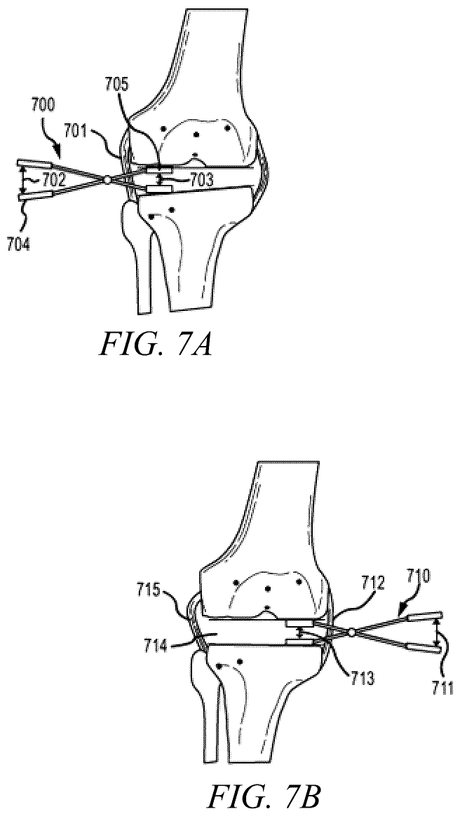

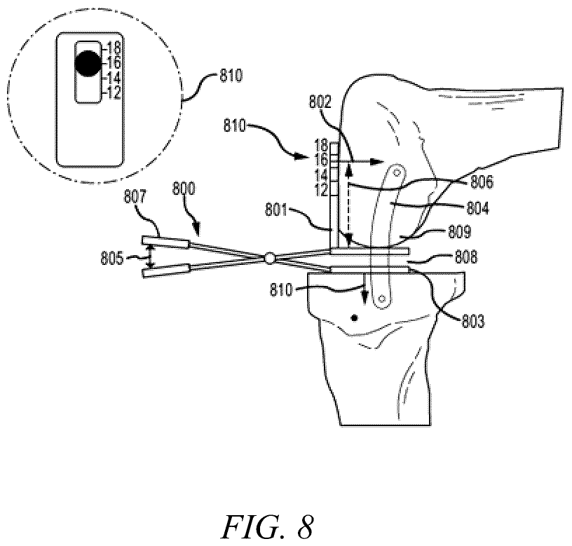

As mentioned above, most surgeons aim to achieve equal and/or symmetrical flexion and extension gaps. FIG. 6 illustrates a knee 600 in 90 Degree flexion after the primary bone cuts have been completed as shown by the femoral distal plane 606 and proximal tibial plane 608. The rotational reference line 603 can be established using two distal holes 602. The distal holes 602 are used subsequently as a reference to assist the surgeon in making the posterior femoral bone resection 605. The initial rotational alignment can be determined using various established surgical techniques, such as posterior condyler or transepicondyler axis represented by the dashed lines 604. Alternatively, a tensioning device, such as the example shown in FIGS. 7-8, can be used to tension the MCL 612 and LCL 613 to measure the flexion gaps prior to adjusting the femoral rotation about the center of knee 601. Typical angular adjustment can range from 0-3+ Degrees 607 internally (clockwise) or externally (counter-clockwise). The rectangular flexion gap 614 can be checked using a spacer block of the same thickness as the combined implant thickness. In this example, the medial 611 and lateral 610 flexion gaps are symmetrical.

In general, establishing the reference planes and femoral rotation can be achieved through various surgical instruments (e.g. manual instruments, computer navigation systems, robotic systems, PSI, and smart instruments) and knee alignments (e.g. mechanical, kinematic, functional, personalized, etc.). Most modern knee implants are designed to be symmetrical in both medial and lateral compartments to match the rectangular and symmetric extension and flexion gaps. However, there are asymmetric implant designs where the medial and lateral gaps are not equal. Also, some clinical studies have found that patients have slightly better clinical outcomes if the gaps are slightly asymmetric mimicking the physiological knee where the lateral gap is slightly larger than the medial gap. For uni-condylar knee replacement, gap and ligament balance are even more critical to achieve good clinical outcomes and implant survivorship by restoring the knee joint line and balancing the ligament tension of the undamaged side. Thus, to achieve optimal clinical outcomes, the gap balancing system should be able to measure knee extension and flexion gaps accurately and transfer such measurements to a robotic-assisted system so that the bone cuts are precise based on the implant design.

FIGS. 7A-7B illustrate one example of extension gap balancing using a manual tensioner 700. The manual tensioner is similar to a lamina spreader such that, when the surgeon squeezes the top and bottom handles 704 by reducing the distance 702, the spreader's paddles 705 inserted between the distal medial femur and proximal medial tibia tensions the LCL 701. The amount of force or load applied is highly dependent on the surgeon's experience and preference. The lateral extension gap 703 can be measured manually using a spacer block of certain thickness, caliper, ruler or any linear measurement tool. This process may be similar for the medial compartment. The manual tensioner 710 is inserted between the distal lateral femur and proximal lateral tibia. By squeezing the handle 704, the distance 711 between the handle is reduced while the distance between the paddle 713 is increased until the LCL 712 is tensioned. The lateral extension gap 714 and LCL 715 are not under tension. There are a couple advantages of measuring the extension gaps independently. First, each compartment can be measured independently, such as during uni-condylar knee replacement surgery. Second, if the goal is to achieve an asymmetric extension gap, this method allows the surgeon to measure the gap independently. A rectangular and symmetric gap can also be measured using the manual tensioner 700, by placing the paddles 705 near the middle of the knee. This has the effect of extension gap and ligament balance.

FIG. 8 shows a lateral view of the knee in 90 Degree flexion. The manual tensioner 800 in this embodiment includes one perpendicular paddle 801 and one parallel paddle 803 instead of two parallel paddles as in the tensioner 700 of FIG. 7. In use, perpendicular paddle 801 contacts the distal femur resection plane and distal reference hole 802 using a nail or spike inserted into bone. Also, the paddle 801 may include a visual mark indicating the distance in millimeters from the distal pin to the horizontal paddle. During a procedure, the horizontal portion of paddle 801 is inserted in the flexion gap and may or may not contact the posterior condyle 809. The horizontal portion of the paddle 801 provides support to the femur bone while the ligament 804 is under tension by squeezing the handle 807 and closing the gap 805. As part of the knee replacement procedure, the anterior cruciate ligament (ACL) is sacrificed during the proximal tibial resection causing anterior subluxation of the tibia as the flexion gap is under tension. To prevent anterior tibia subluxation, which can result in inaccurate flexion gap measurements, the parallel paddle 803 contacts the proximal tibial reference plane and is secured to the bone using a spike or pin 810 inserted into the proximal holes 306. The flexion gap measurement considers the distance 806 from the distal hole 802 to the parallel paddle plus the gap 808, which can be measured using a spacer block, caliper, ruler, or any linear measurement tool. The distance 806 is provided by the visual marks 810 in 1- or 2-mm increments. The manual tensioner can thus measure the flexion gap for each compartment (medial or lateral) independently or together if rectangular gap is desired by using a wider perpendicular paddle that accommodates both medial and lateral condyles and two distal holes.

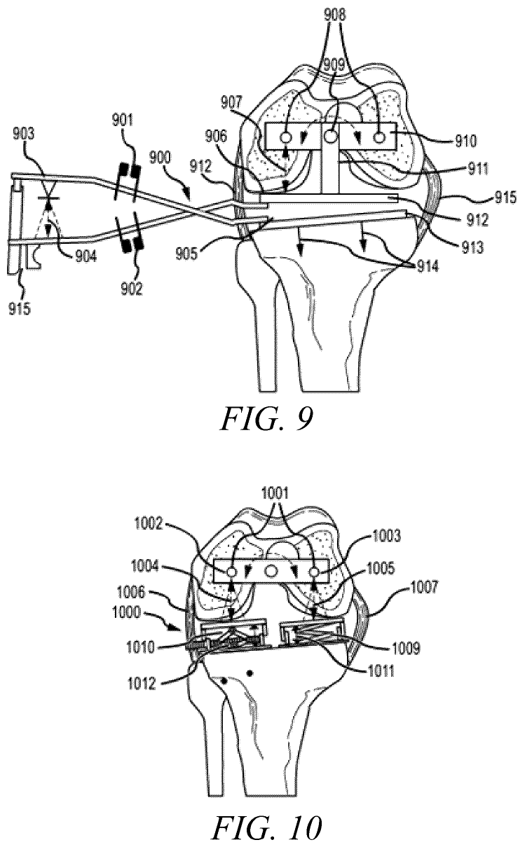

FIG. 9 illustrates a knee in 90 Degrees of flexion. Attached to the distal femur reference plane and rotational reference holes 302 is a thin rectangular plate 910, which may be about 2 to 4 mm with two spikes 908 inserted into bone in some instances. At the center of the plate 910 is a rotational hinge 909, which also represents the center of the femoral component 501. Connected to the hinge 909 is a T-shaped beam, which consists of a vertical beam 911 and horizontal beam/plate 912. Attached to the proximal tibial reference plane is a thin plate 913, which may be about 2 to 4 mm with one or two spikes 914 inserted into proximal reference holes 302 in some implementations. In one embodiment, plate 913 has no spikes 914 where the surgeon is manually preventing anterior tibia subluxation. A manual tensioner 900 like tensioner 700 described above can be used to tension the LCL 912 by inserting the spreader 914 between the horizontal plates 912 and 913. The plates protect the soft cortical bone from damage potentially caused by the spreader paddle. When the surgeon squeezes the tensioner handles, the horizontal plate 912 contacts the lateral posterior condyle at location 906 rotating about the hinge 909. The angular rotation about the hinge can be measured using a visual dial indicator or sensors, such as potentiometer, encoder, accelerometer, to name a few. The lateral flexion gap 905 measurement takes into consideration the distance 907, plate thicknesses of 912 and 913, and gap 905. Unlike the tensioner in 700 where the gap is measured manually, tensioner 900 may include various sensors to measure the gap (millimeters) and load (Newton) of the ligaments. Located near the handle is a distance measuring sensor 903, which can be optical (visible or infrared), ultrasonic, or linear potentiometer. The distance measurement 904 is proportional to the flexion gap 905 since the dimension and geometry of the tensioner 900 is well defined. Also, attached to the top and bottom handles are load cells 901 and 902 that measure the total load being applied to tension the ligament. A locking mechanism 915 can be used to lock the handles at the desired tension. Similarly, the medial flexion gap and load can be measured using the manual tensioner 900 by inserting the spreader between the horizontal plates 912 and 913 to tension the MCL 915.

In one embodiment, the manual tensioner 900 is placed near the center of the knee between the horizontal plates 912 and 913. When the desired load has been applied by squeezing the handles, both the LCL 912 and MCL 915 will be balanced, creating a rectangular flexion gap. Any rotation about the hinge 900 and gap 905 is measured by the distance sensor 903 and angular sensor 909. These measurements can be used to adjust the posterior cut 605 to achieve the desired rectangular flexion gap 614. Similarly, the extension gap can be measured, and medial/lateral ligaments balanced, by inserting the spreader between the distal plate 910 and proximal plate 913. If the goal is to achieve symmetric extension and flexion rectangular gaps and/or balanced ligaments, then measuring both compartments at the same time will achieve similar results as measuring each compartment individually for total knee replacement surgery.

FIG. 10 illustrates a knee in 90 Degrees of flexion with mechanical tensioner for ligament and gap balance 1000. A rectangular plate like that illustrated in FIG. 9 is attached to the distal femur plane 301 and rotational holes 302. Located near the two distal holes 1002 and 1003 are distance measuring sensors 1001 that measure the distance 1004 and 1005 from the rotational reference line to the ligament tensioning device for the medial and lateral compartments, respectively. For the medial compartment, the mechanical tensioner 1009 is spring loaded with a certain spring constant sufficient to tension the MCL 1007 at a desired load. The medial flexion gap 1011 can be measured using any manual measuring tool or distance measuring sensor. In use, the device 1009 is inserted between the medial posterior condyle and proximal tibia where the top and bottom plate contacts the bone. For the lateral compartment, the mechanical tensioner 1010 is a jack-type device that can be raised and lowered by turning an operational screw manually or by a motorized actuator until the desired load is reached. Various sensors can be used to measure the load and distance as mentioned above. Similarly, the device 1010 is inserted between the lateral posterior condyle and proximal tibia. Although only two examples are given regarding the mechanical mechanism to acuate the jack-type device, there are many other electro-mechanical or non-mechanical actuators, such as air or water bags, hydraulic pistons, and linear actuators (manual or motorized) to name a few that may be utilized with the device. Also as mentioned above for manual tensioners, the mechanical tensioners here can balance both ligaments 1006-1007 and measure flexion gap (rectangular) simultaneously by using one tensioner that covers both medial and lateral condyles. Similarly, the extension gap can be measured individually or together as a rectangular gap using one or two tensioners.

Clinical studies have shown that the extension gap may vary <1 mm for loads between 100 N to 200 N regardless of whether the ACL or PCL are sacrificed or not. Conversely, the flexion gap may vary 4-6 mm for loads between 100 to 200 N when the ACL and PCL are sacrificed versus 1-2 mm for ACL sacrificed only. Thus, rotational (I/E) and A/P femoral component adjustments are important in achieving the desired flexion gap and ligament balance for optimal clinical outcome. FIGS. 11-14 are frontal and axial views of a bone mounting device that allows rotational adjustments for a 2-axis robotic-assisted system for secondary bone cuts to achieve desired flexion gap and/or ligament balance.

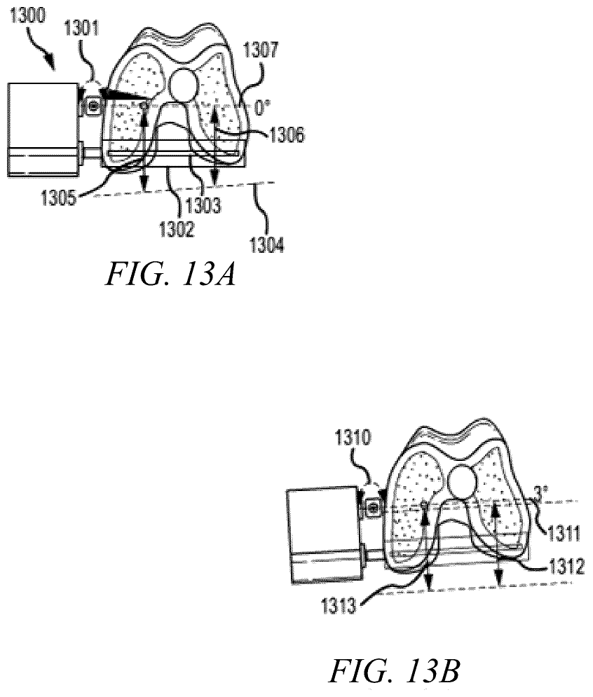

Starting with FIG. 11 is a frontal view of the femur bone with a mounted rotational adjustment device 1100 for the robotic-assisted system shown in FIG. 13. Using the same distal reference plane 301 and distal drill holes 302 as the gap balancer system above, the drill guide plate 1102 may be secured to the femur using two pins 1101, which allows for a drill bit 1103 to be inserted into the medial femur parallel to the reference plane with a certain distal offset 1107. After the drill bit 1103 has been inserted into the bone, the drill guide plate 1102 and pins 1101 are removed from the bone. Next, a portion of the rotational adjustment mechanism or hinge 1106 is attached to the drill bit 1103 while the robot 1105 is attached to the other portion of the hinge 1104. A set screw 1108 can be used to secure the hinge 1100 by clamping plate 1104 and 1106 resulting in a friction lock. Note, only the robot is allowed to rotate about the center of hinge 1108 while the pin 1103 is fixed to the bone as the initial reference position for the implant's position and orientation.

Continue with FIG. 12 showing the robot's rotational adjustment mechanism 1200 attached to pin 1205 near or anterior of the medial transepicondyles 1208. The center of the robot's rotational adjustment or hinge is located at location 1206 and can rotate either clockwise or counterclockwise at any angle 1202. In some cases, the femoral rotational adjustment angle (0) 1204 is in the range of (−5 to +5 Degree) in 1 or 2 Degrees increments from the rotational reference line 302. Also attached to the hinge 1200 is a portion of the robot 1209 as shown in FIG. 13. To prevent the pin 1205 from loosening or walking due to the vibration of the oscillation saw, a fixation arm 1203 attached to the hinge 1106 is secured to one or both distal holes 1201 and 1207 using spikes inserted into bone. Also, the length of arm 1203 from hinge 1206 to either distal hole 1201 or 1207 may be known to calculate any A/P offset between center of hinge 1206 and center of femoral component or knee 909. The formula to calculate the Robot's A/P offset can be defined using simple a right-angle formula as follows:

Robot A/P offset=distance from hinge to center of knee (mm)*Sine (θ)

The A/P offset varies depending on the rotational angle (θ). Any A/P offset between pin 1205, arm 1203 and implant (A/P) adjustment are also included in the total A/P offset of the robot to ensure accurate placement of the femoral component 1400.

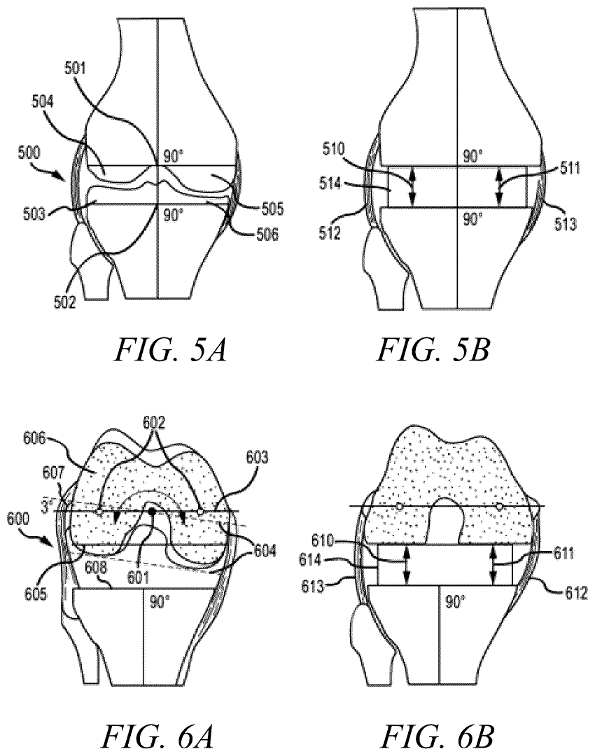

FIGS. 13A-13B illustrate a 2-axis robot 1300 attached to rotational adjustment mechanism 1200 secured to the bone using pin 1205 and arm 1203 while holding a cutting guide 1302 with slot 1303 for use with a power oscillating saw system. Initially, the rotational angle (0) 1301 is set at 0 Degree, which is the default reference line 1307. As described in the flowchart of FIG. 4 and gap balancer system in FIGS. 5-10, the extension and flexion gaps can be measured using either a manual or dynamic knee balancer device for one or both medial and lateral compartments. Once the extension and flexion gaps have been measured and recorded, whether the desired flexion gap is symmetric or asymmetric gap, implant size, rotation (I/E) 1301, medial 1305 (A/P) offset, and lateral 1306 (A/P) offset can be adjusted using hinge 1301 and forward kinematics of 2-axis robot 1409. FIG. 13B illustrates the rotational adjustment of three Degrees 1310 relative to the robot's hinge. For this example, the desired goal is a symmetric and balanced extension and flexion gaps for both medial 1313 and lateral 1312 compartments. The robot's A/P offset 1311 is calculated using the equation described above so saw guide 1302 assists the surgeon in resecting the exact amount of posterior bone 1405.

FIG. 14 is a lateral view of the knee with robot 1300 mounted near the transepicondyle using the rotational adjustment mechanism described in FIGS. 11-13. The robot comprises a 2-rotational axis 1407 and 1410 which defines the workspace 1409 relative to X-Y axes at 1407. Any angle within the robot's workspace 1409 can be reached by cutting guide 1302 by adjusting the rotational angle of axis 1 at 1407 and axis 2 at 1410. For most modern implant design 1400, the distal 1401, anterior 1402, posterior 1405, anterior chamfer 1404 and posterior chamfer 1403 bone cuts can be defined by a 2-axis plane. The robot 1300 can accommodate the implant position and orientation adjustments based on the gap balancing system and distance 1408. For example, I/E can be adjusted using 1200, and A/P, F/E and implant size can be adjusted by the robot 1300.

As mentioned above, the distal holes 302 may be used as the reference line for both the robot and implant rotational adjustment. Subsequently, any A/P offset based on the desired extension and flexion gaps can be easily determined by the robot. An advantage of using the distal line as reference in certain implant designs are anterior referencing versus posterior referencing. For example, the distance 1408 can vary with implant size for anterior referencing systems versus fixed for posterior referencing implants. By using the same reference planes (301 and 303) and lines (302 and 306) for both gap balancer and implant system, the surgeon may use any manual, robotic-assisted surgical systems, and/or computer navigation systems.

Thus far, the gap balancer system has been focused on the extension/flexion gaps and MCL/LCL for the femoral and tibial components. Moreover, as part of the total knee and patella-femoral replacement surgeries, the patella is also replaced by a plastic button of similar shape, thickness and size as the original bone removed. FIGS. 15A-15B show the patella component 1500 and the various degrees of freedom (3-translations and 3-rotations) 1501. A balanced patella-femoral joint is also crucial in eliminating patella pain and loosening to improve the clinical outcomes and implant survivorship. To balance the patella-femoral joint, a 3-axis load cell 1601 may be embedded in the patella button 1600 to provide load and gap information. The patella button 1600 consists of a dome like surface 1606, base 1607 and two or more pegs 1603. After the patella has been prepared by removing the arthritic bone in FIG. 15A, one or more pegs are drilled into the distal bone using a template. Location and depth of the holes for the pegs may be pre-determined using the cutting plane as reference. The pegs on the patella button 1600 are then inserted into the matching hole on the bone to provide a reference position and orientation, as well as fixation (cemented or cementless).

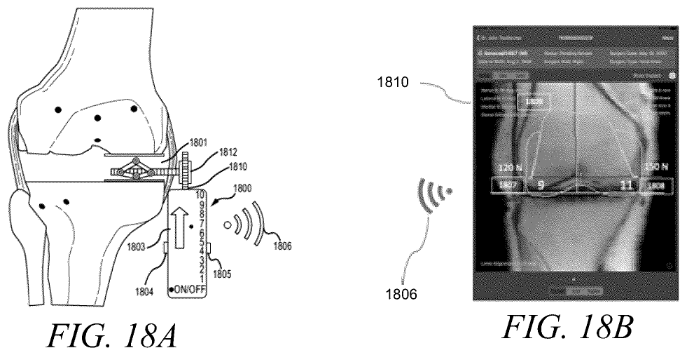

Inside the button 1600 is a load cell 1601 and electronics (PCB) 1602. The load cell 1601 is made up of 4 or more deformable beams shown in 1605. The center of the beams is a circular mass 1608 where the origin of the 3-axis (x, y, z) is defined. Each of the beam consists of one or more load cells 1612, like 901-902, are mounted along each of the (x, y, z) axis. Force (F) and inertial (M) can be calculated using load cells along any of the 3-axis (x, y, z). The beams are also attached to a rectangular or circular case 1608 secured to the baseplate 1607 through the PCB 1602. One or more posts 1604 is attached to the mass 1601 to sense any load from the patella button during trialing. The patella button 1600 can be single-use or reusable and may or may not contain any power source as a battery. Sensor data from the load cells can be transmitted wirelessly to a computer system in FIG. 18B where real-time measurements are displayed. Based on the real-time measurement and surgeon preferences, various patella button sizes and shapes shown in 1502 can be used to optimize the knee kinematics for each patient described below.

FIGS. 17A-17B illustrate an example of a dynamic total knee gap balancing system for all 3 compartments (femur 1702, tibia 1703 and patella 1701). A similar balancing system can also be developed for uni-knee or bi-compartment, or patella femoral replacement surgeries. Ideally, the shape and size of the implants are the same as the final implanted design to ensure the data measured is as close to final as possible. After the initial bone resections are completed using the PSI system described in FIGS. 1-2 including the patella, system 1700 can be used to measure the gaps and load of the ligaments for all compartments individually or together using a manual tensioning system 900 or dynamic spacers 1000. In one embodiment, the distal plate 910 and beams 911-912 are replaced by a near shape femoral component 1400 standard off-the-shelf or custom. Since the secondary cuts have not been performed, the dynamic system 1700 allows for real-time data measurements and adjustments. Based on the surgeon preferences, the desired implant position and orientation can be recorded or transmitted wirelessly to the robot 1300 or displayed on a computer monitor shown in FIG. 18B. As mentioned above, the extension and flexion gaps can be symmetric or asymmetric based on the surgeon preferences and implant design. FIG. 17B shows the medial 1704 and lateral 1705 gap measurements performed individually with I/E rotation fixed at 0 Deg 1707. If a symmetric flexion gap is the goal, then a rectangular dynamic spacer can be used to tension both LCL and MCL allowing the femur to rotate about 1706. At the same time, the F/E of the femoral component can be adjusted using a hinge that allows angular adjustment in the lateral plane 1707 like 1407. Finally, the patella button sensor 1600 can be used to measure and adjust the shape and size of the patella button to achieve an optimal balanced knee.

FIG. 18A depicts a motorized gap balancer 1800 to balance the medial extension gap. For simplicity, the motorized actuator has an up 1804 and down 1805 button that allows the user to adjust the gap 1801 using a perpendicular lead screw 1802 and hinged jack. The perpendicular lead screw 1802 can be a planetary or worm gear to increase torque applied to the jack. Rotational sensors or load cells can be used in the gear 1802 to measure the load and gap. The measurement data can be displayed on 1800 using a LED or digital display 1803 or can be transmitted to a computer shown in FIG. 18b wired or wirelessly 1806 using Bluetooth, Wifi, Cellular or Serial communication. The surgical planning software 1810 can display the initial implant position and orientation 1809 and the actual measured medial 1808 and lateral 1807 gaps, as well as the load data for the MCL and LCL. Using soft tissue analysis algorithms, the planning software 1810 can calculate the optimal implant position and orientation based on the surgeon preferences and implant design. Any real-time adjustments of the implant position can be performed using the two-axis robot 1300 and rotational adjustment mechanism 1200.

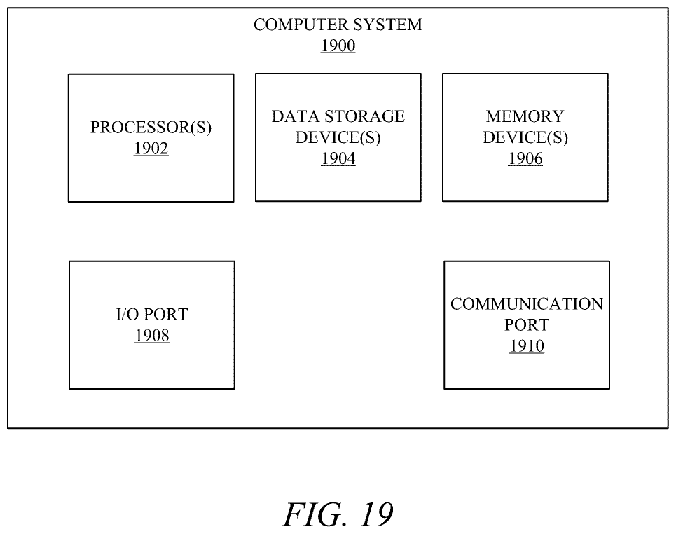

Referring to FIG. 19, a detailed description of an example computing system 1900 having one or more computing units that may implement various systems and methods discussed herein is provided. It will be appreciated that specific implementations of these devices may be of differing possible specific computing architectures not all of which are specifically discussed herein but will be understood by those of ordinary skill in the art.

The computer system 1900 may be a computing system capable of executing a computer program product to execute a computer process. Data and program files may be input to the computer system 1900, which reads the files and executes the programs therein. Some of the elements of the computer system 1900 are shown in FIG. 19, including one or more hardware processors 1902, one or more data storage devices 1904, one or more memory devices 1908, and/or one or more ports 1908-1910. Additionally, other elements that will be recognized by those skilled in the art may be included in the computing system 1900 but are not explicitly depicted in FIG. 19 or discussed further herein. Various elements of the computer system 1900 may communicate with one another by way of one or more communication buses, point-to-point communication paths, or other communication means not explicitly depicted in FIG. 19.

The processor 1902 may include, for example, a central processing unit (CPU), a microprocessor, a microcontroller, a digital signal processor (DSP), and/or one or more internal levels of cache. There may be one or more processors 1902, such that the processor 1902 comprises a single central-processing unit, or a plurality of processing units capable of executing instructions and performing operations in parallel with each other, commonly referred to as a parallel processing environment.

The computer system 1900 may be a conventional computer, a distributed computer, or any other type of computer, such as one or more external computers made available via a cloud computing architecture. The presently described technology is optionally implemented in software stored on the data stored device(s) 1904, stored on the memory device(s) 1906, and/or communicated via one or more of the ports 1908-1910, thereby transforming the computer system 1900 in FIG. 19 to a special purpose machine for implementing the operations described herein. Examples of the computer system 1900 include personal computers, terminals, workstations, mobile phones, tablets, laptops, personal computers, multimedia consoles, gaming consoles, set top boxes, and the like.

The one or more data storage devices 1904 may include any non-volatile data storage device capable of storing data generated or employed within the computing system 1900, such as computer executable instructions for performing a computer process, which may include instructions of both application programs and an operating system (OS) that manages the various components of the computing system 1900. The data storage devices 1904 may include, without limitation, magnetic disk drives, optical disk drives, solid state drives (SSDs), flash drives, and the like. The data storage devices 1904 may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory devices 1906 may include volatile memory (e.g., dynamic random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in the data storage devices 1904 and/or the memory devices 1906, which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

In some implementations, the computer system 1900 includes one or more ports, such as an input/output (I/O) port 1908 and a communication port 1910, for communicating with other computing, network, or reservoir development devices. It will be appreciated that the ports 1908-1910 may be combined or separate and that more or fewer ports may be included in the computer system 1900.

The I/O port 1908 may be connected to an I/O device, or other device, by which information is input to or output from the computing system 1900. Such I/O devices may include, without limitation, one or more input devices, output devices, and/or environment transducer devices.

In one implementation, the input devices convert a human-generated signal, such as, human voice, physical movement, physical touch or pressure, and/or the like, into electrical signals as input data into the computing system 1900 via the I/O port 1908. Similarly, the output devices may convert electrical signals received from computing system 1900 via the I/O port 1908 into signals that may be sensed as output by a human, such as sound, light, and/or touch. The input device may be an alphanumeric input device, including alphanumeric and other keys for communicating information and/or command selections to the processor 1902 via the I/O port 1908. The input device may be another type of user input device including, but not limited to: direction and selection control devices, such as a mouse, a trackball, cursor direction keys, a joystick, and/or a wheel; one or more sensors, such as a camera, a microphone, a positional sensor, an orientation sensor, a gravitational sensor, an inertial sensor, and/or an accelerometer; and/or a touch-sensitive display screen (“touchscreen”). The output devices may include, without limitation, a display, a touchscreen, a speaker, a tactile and/or haptic output device, and/or the like. In some implementations, the input device and the output device may be the same device, for example, in the case of a touchscreen.

The environment transducer devices convert one form of energy or signal into another for input into or output from the computing system 1900 via the I/O port 1908. For example, an electrical signal generated within the computing system 1900 may be converted to another type of signal, and/or vice-versa. In one implementation, the environment transducer devices sense characteristics or aspects of an environment local to or remote from the computing device 1900, such as, light, sound, temperature, pressure, magnetic field, electric field, chemical properties, physical movement, orientation, acceleration, gravity, and/or the like. Further, the environment transducer devices may generate signals to impose some effect on the environment either local to or remote from the example computing device 1900, such as, physical movement of some object (e.g., a mechanical actuator), heating or cooling of a substance, and/or the like.

In one implementation, a communication port 1910 is connected to a network by way of which the computer system 1900 may receive network data useful in executing the methods and systems set out herein as well as transmitting information and network configuration changes determined thereby. Stated differently, the communication port 1910 connects the computer system 1900 to one or more communication interface devices configured to transmit and/or receive information between the computing system 1900 and other devices by way of one or more wired or wireless communication networks or connections. Examples of such networks or connections include, without limitation, Universal Serial Bus (USB), Ethernet, Wi-Fi, Bluetooth®, Near Field Communication (NFC), Long-Term Evolution (LTE), and so on. One or more such communication interface devices may be utilized via the communication port 1910 to communicate one or more other machines, either directly over a point-to-point communication path, over a wide area network (WAN) (e.g., the Internet), over a local area network (LAN), over a cellular (e.g., third generation (3G) or fourth generation (4G) or fifth generation (5G) network), or over another communication means. Further, the communication port 1910 may communicate with an antenna or other link for electromagnetic signal transmission and/or reception.

In an example implementation, the MLA system 102, and software and other modules and services may be embodied by instructions stored on the data storage devices 1904 and/or the memory devices 1906 and executed by the processor 1902. The computer system 1900 may be integrated with or otherwise form part of the MLA system 102.

The system set forth in FIG. 19 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations together with all equivalents thereof.

While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Without intent to limit the scope of the disclosure, examples of instruments, apparatus, methods and their related results according to the embodiments of the present disclosure are given herein. Note that titles or subtitles may be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

1. A system for knee replacement surgery, comprising:
a sensor measuring a distance of a gap in a knee joint during a knee replacement procedure;
a tensioner device providing tension to the medial and lateral collateral ligaments based on the distance measurement obtained from the sensor;
a computing system displaying real-time data from the sensor on a display in communication with the computing system; and
a robotic surgical device shaping a distal end of a femur based on the gap information and on a patient-specific knee instrument mated to the femur to provide an initial position and orientation of a knee implant;
wherein the computing system determines a recommended implant design and an implant size based on dynamic knee measurements received from the sensor and the tensioner device during the knee replacement procedure.

2. The system of claim 1, wherein the sensor comprises at least one of a force sensor, an ultrasonic sensor, a pressure sensor, or an optical sensor.

3. The system of claim 1, wherein the tensioner device comprises a manual spreader.

4. The system of claim 1, wherein the tensioner device comprises a spring-loaded spreader.

5. The system of claim 1, wherein the tensioner device comprises a motorized linear actuator.

6. The system of claim 1, wherein the sensor measures an extension gap and a flexion gap of the knee joint during the knee replacement procedure, wherein the extension gap is measured at a zero degrees orientation of the knee joint and the flexion gap is measured at a 90 degree orientation of the knee joint.

7. The system of claim 1, wherein the sensor measures the gaps and the tensioner device measures ligament tension dynamically throughout a range of motion of the knee joint from 0 degrees to more than 120 degrees of rotation.

8. The system of claim 1, further comprising a communication interface between the sensor and the computing system, wherein the communication interface is a wired or a wireless communication interface.

9. The system of claim 8, wherein the wireless communication interface is a Bluetooth connection, an RFID connection, or a Wi-Fi connection.

10. The system of claim 1, wherein the computer system displays a preoperative planning data simultaneously with a real-time measurement from the sensor.

11. A method for knee replacement surgery, the method comprising:
obtaining a preoperative two-dimensional imaging scan of a patient's knee joint for preoperative planning of the knee replacement surgery;
manufacturing a patient-specific instrument for knee replacement surgery based on the preoperative planning;
resecting, utilizing the patient-specific instrument, a femur and a tibia of the knee joint;
measuring a gap between the resected femur and the resected tibia of the knee joint at 0 and 90 degrees using a sensor device;
determining a position and an initial size of one or more knee implants based on the measured gaps;
sending the position and the initial size of the one or more knee implants to a robotic surgical system;
shaping, based on the position and the size of the one or more knee implants, the distal end of the femur with the robotic surgical system; and
receiving, from a computing device and based on the measured gap from the sensor device, an updated implant size and implant shape based on the measured gap received from the sensor device during the shaping of the distal end of the femur.

12. The method of claim 11, wherein the sensor device comprises at least one of a force sensor, a pressure sensor, and ultrasound sensor, or an optical sensor.

13. The method of claim 11, wherein the sensor device measures an extension gap and a flexion gap of the knee joint, wherein the extension gap is measured at a zero degrees orientation of the knee joint and the flexion gap is measured at a 90 degree orientation of the knee joint.

14. The method of claim 11, wherein the sensor device measures the gaps dynamically throughout a range of motion of the knee joint from 0 degrees to more than 120 degrees of rotation.

15. The method of claim 11, further comprising displaying, on a display device associated with the computing device, real-time measurement data along with preoperative planning data during knee replacement surgery.

16. The method of claim 11, further comprising utilizing the patient-specific knee instrument to determine an initial position and an orientation of the one or more knee implants.

17. The method of claim 11, further comprising using a tensioner device to measure tension in a medial and a lateral collateral ligament of the patient's knee joint.

18. The method of claim 17, wherein the tensioner device comprises a manual spreader.

19. The method of claim 17, wherein the tensioner device comprises a spring-loaded mechanism.

20. The method of claim 17, wherein the tensioner device comprises a motorized linear actuator.
